(19) United States
(12) Patent Application Publication
(10) Pub. No.: US 2016/0015934 A1
(43) Pub. Date: Jan. 21, 2016

(54) SHEATH

(71) Applicant: TERUMO KABUSHIKI KAISHA, Tokyo (JP)

(72) Inventor: Ryo OKAMURA, Fujinomiya-shi (JP)

(73) Assignee: TERUMO KABUSHIKI KAISHA, Tokyo (JP)

(21) Appl. No.: 14/868,832

(22) Filed: Sep. 29, 2015

Related U.S. Application Data
(63) Continuation of application No. PCT/JP2013/059919, filed on Apr. 1, 2013.

(51) Int. Cl. A61M 25/01; A61M 25/00; A61M 25/09

(52) U.S. Cl. CPC A61M 25/01; A61M 25/09; A61M 25/0023; A61M 2025/0062

(57) ABSTRACT

At least one distal side slope path is formed by penetrating an outer peripheral wall in the vicinity of a distal portion of a sheath main body and at least one proximal side slope path is formed by penetrating an outer peripheral wall in the vicinity of a proximal portion of the sheath main body. The at least one distal side slope path is inclined onto a proximal side from the outer peripheral surface to the inner peripheral surface of the sheath main body and the at least one proximal side slope path is inclined onto a distal side from the outer peripheral surface to the inner peripheral surface of the sheath main body. Therefore, blood is made to flow into a lumen and is made to flow out from the lumen along the blood flow in a blood vessel.

This application is a continuation of International Application No. PCT/JP2013/059919 filed on Apr. 1, 2013, the entire content of which is incorporated herein by reference.

### TECHNICAL FIELD

The invention generally relates to a sheath, and more particularly to an introducer sheath used for percutaneously guiding a catheter into a blood vessel.

### BACKGROUND DISCUSSION

In the medical field, a catheter which can be inserted into a diseased region along a blood vessel has been used to treat the diseased region in the blood vessel. In general, the catheter is inserted into a predetermined position in a blood vessel through a lumen in an introducer sheath, which has been indwelled in the blood vessel, and is inserted into a vicinity of a diseased region along the blood vessel as it is when being led out to the inside of the blood vessel from a distal end opening portion at a distal end of the introducer sheath. In this manner, it is possible to easily secure a blood vessel and to prevent friction between a catheter and the skin when treating a diseased region using the catheter by inserting the catheter into the blood vessel through an introducer sheath.

Here, the introducer sheath has an elongated shape in order to be inserted into a blood vessel, and there is a problem in that slidability of the catheter is impaired due to contact of a part of the inner peripheral surface of the introducer sheath with the catheter. For this reason, in some cases, it is impossible to promptly insert a catheter into a blood vessel, for example, an operator feels resistance on hand during a surgical procedure using the catheter. Particularly, a large amount of time is required to insert the catheter into the diseased region when using a long introducer sheath.

A technique has been developed for improving slidability of a catheter by coating the inner peripheral surface of an introducer sheath with a hydrophilic polymer which exhibits lubricity through wetting (absorbing water). Here, it is necessary to wet the hydrophilic polymer in blood by making blood flow into a lumen of the introducer sheath in order for the hydrophilic polymer to exhibit lubricity. However, since a distal portion of the introducer sheath has a shape reduced in diameter so as to improve penetrating properties of the introducer sheath into a living body, there is almost no gap between a distal end opening portion and a catheter. Therefore, only a slight amount of blood can flow into the lumen from the distal end opening portion. For this reason, even if the inner peripheral surface of the introducer sheath is coated with a hydrophilic polymer, it is impossible to supply sufficient blood to the hydrophilic polymer. Therefore, the current state is that it is impossible to improve lubricity of the inner peripheral surface of the introducer sheath, and thus, it is impossible to improve the slidability of the catheter.

For example, Japanese Application Publication No. 11-299897 discloses an introducer sheath which makes blood in a blood vessel flow into a lumen by providing a side hole on a side wall. Accordingly, it is possible to make blood flow into the lumen without passing through the distal end opening portion of the introducer sheath.

### SUMMARY

However, a side hole which is formed on a side wall is formed for the purpose of making blood flow into a part of a lumen in order to measure blood pressure and an electrocardiogram, and is not for circulating blood over a wide range in the lumen. For this reason, blood is not supplied to the entirety of a hydrophilic polymer even if the inner peripheral surface of the introducer sheath is coated with the hydrophilic polymer. Therefore, it is impossible to improve slidability of a catheter significantly.

The sheath disclosed here is constructed in a way allowing the sheath to circulate blood over a wide range in a lumen.

A sheath includes: a sheath main body which is elongated in order to be inserted into a blood vessel; a lumen which is formed in the sheath main body; at least one distal side slope path which is formed by penetrating an outer peripheral wall in the vicinity of a distal portion of the sheath main body; and at least one proximal side slope path which is formed by penetrating an outer peripheral wall in the vicinity of a proximal portion of the sheath main body, in which in order to make blood flow into the lumen and to make blood flow out from the lumen along the blood flow in a blood vessel, the at least one distal side slope path is formed so as to be inclined onto a proximal side from the outer peripheral surface to the inner peripheral surface of the sheath main body and the at least one proximal side slope path is formed so as to be inclined onto a distal side from the outer peripheral surface to the inner peripheral surface of the sheath main body.

The inner peripheral surface of the sheath main body is preferably coated with a hydrophilic polymer which exhibits lubricity by being wetted by blood which has flowed into the sheath main body.

Also, a spiral groove is preferably formed on the inner peripheral surface of the sheath main body such that the blood which has flowed into the lumen circulates while drawing a spiral orbit.

Also, at least either of the distal side slope path and the proximal side slope path may be formed so as to be further inclined in a circumferential direction of the sheath main body along the groove.

According to another aspect, a sheath comprises an elongated sheath main body insertable into a blood vessel and possessing a distal portion and a proximal portion, with the sheath main body possessing an outer peripheral surface. The sheath main body includes a lumen passing completely through the lumen, wherein the sheath main body includes an inner peripheral wall surrounding the lumen. A first through hole passes through the outer peripheral wall of the sheath main body so that the first through hole opens to the outer peripheral surface of the sheath main body and opens to the inner peripheral surface of the sheath main body, with the first through hole opening to the outer peripheral surface of the sheath main body at a location more distal of a location at which the first through hole opens to the inner peripheral surface of the sheath main body. A second through hole passes through the outer peripheral wall of the sheath main body so that the second through hole opens to the outer peripheral surface of the sheath main body and opens to the inner peripheral surface of the sheath main body, with the second through hole opening to the outer peripheral surface of the sheath main body at a location more proximal of a location at which the second through hole opens to the inner peripheral surface of the sheath main body. The first through hole is located distal of the second through hole.

As a method of using the sheath, the above-described sheath is indwelled in a blood vessel, a catheter is introduced into the blood vessel through a lumen formed in a sheath main body, and blood is made to flow into the lumen and to flow out from the lumen along the blood flow in a blood vessel by at least one distal side slope path which is formed so as to be inclined onto a proximal side from the outer peripheral surface to the inner peripheral surface in the vicinity of a proximal portion of the sheath main body and by at least one proximal side slope path which is formed so as to be inclined onto a distal side from the outer peripheral surface to the inner peripheral surface in the vicinity of a proximal portion of the sheath main body. In the method disclosed here, the sheath is inserted into a blood vessel and is moved within the blood vessel so that blood in the blood vessel either enters the lumen by way of the distal side slope path and exist the lumen by way of the proximal side slope path, or enters the lumen by way of the proximal side slope path and exits the lumen by way of the distal side slope path.

According to the sheath and method disclosed here, blood is made to flow into the lumen and to flow out from the lumen along the blood flow in a blood vessel, and therefore, it is possible to circulate blood over a wide range in the lumen.

### BRIEF DESCRIPTION OF DRAWINGS

FIG. 1 is a plan view showing a configuration of an introducer sheath using a sheath main body according to Embodiment 1 of the invention.

FIG. 2 is a cross-sectional view showing a configuration of the sheath main body according to Embodiment 1.

FIG. 3 is a view showing a state in which the introducer sheath is indwelled in a blood vessel.

FIG. 4 is a view showing a state in which a catheter is introduced into the blood vessel through the introducer sheath.

FIG. 5 is a cross-sectional view showing a configuration of a sheath main body according to Embodiment 2.

FIG. 6 is a cross-sectional view showing a configuration of a sheath main body according to a modification example of Embodiment 2.

FIG. 7 is a cross-sectional view taken along line A-A in FIG. 6.

FIG. 8 is a cross-sectional view showing a state in which a sheath main body is disposed such that a distal side of the sheath main body becomes downstream with respect to a blood flow in a blood vessel.

FIG. 9 is a cross-sectional view showing a configuration of the sheath main body in which a distal side slope path is formed in the vicinity of a proximal portion and the central portion.

FIG. 10 is a view showing a configuration in the vicinity of a distal portion of the sheath main body in which a circular opening portion is formed.

FIG. 11 is a view showing a configuration in the vicinity of the distal portion of the sheath main body in which a crescent-shaped opening portion is formed.

### DETAILED DESCRIPTION

Hereinafter, embodiments representing examples of the inventive sheath disclosed herewill be described based on the accompanying drawings. Features that are common to different embodiments are identified by the same reference numerals throughout and so detailed descriptions of already described features will not be repeated. In some cases, dimensional ratios in the drawings are exaggerated and are different from the actual ratios for the convenience of description.

An introducer sheath is a device for securing an access route to the inside of a lumen in a living body. In the description below, the hand operation unit side of the device (i.e., the end of the device operated/held by the operator) will be referred to as a “proximal side” or “proximal end”, and the side through which the device is inserted into the lumen in a living body (i.e., the end of the device first inserted into the lumen in the living body) will be referred to as a “distal side” or “distal end”.

### Embodiment 1

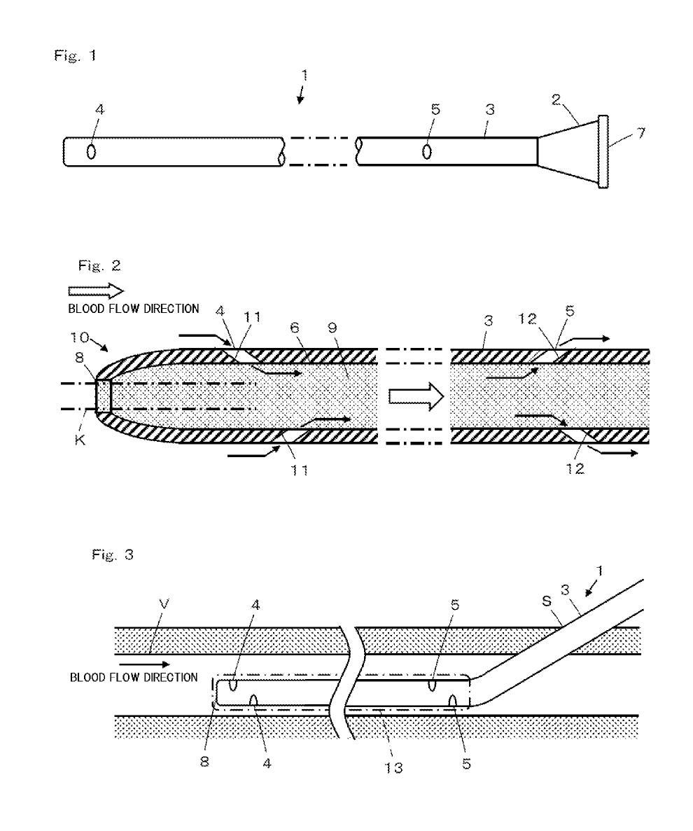

FIG. 1 shows a configuration of an introducer sheath provided with a sheath main body according to Embodiment 1 disclosed here. The introducer sheath 1 is provided for introducing a catheter into a blood vessel and has a sheath main body 3 which is inserted into the blood vessel; and a hub 2 which is disposed at a proximal end of the sheath main body 3.

The sheath main body 3 has an elongated form in order to be inserted into a blood vessel. On the outer peripheral surface of the sheath main body 3, an elliptic (elliptically-shaped) opening portion 4 is formed in the vicinity of a distal portion of the sheath main body 3 and an elliptic (elliptically-shaped) opening portion 5 is formed in the vicinity of a proximal portion of the sheath main body 3. In addition, as shown in FIG. 2, a lumen 6 extending from a proximal end to a distal end is formed in the sheath main body 3, a proximal side (proximal end) of the lumen 6 is connected to a port 7 which is formed in the hub 2, and a distal side (distal end) of the lumen 6 is connected to a distal end opening portion 8 which is formed at a distal end of the sheath main body 3. Accordingly, it is possible to lead a catheter K, which has been introduced from the port 7 of the hub 2, from the distal end opening portion 8 to the outside through the lumen 6. In addition, a hemostasis valve which is formed of an elastic member is liquid-tightly fixed to the hub 2 which is configured such that blood does not leak after the introducer sheath is indwelled in a blood vessel. The hub 2 may be provided with, for example, a side port such that a liquid such as physiological saline or the like can be injected into the introducer sheath.

In addition, the inner peripheral surface of the sheath main body 3 is coated with a hydrophilic polymer 9 which exhibits lubricity when wetted by blood which has flowed into the sheath main body. Examples of the hydrophilic polymer 9 include a cellulose-based polymer material, a polyethylene oxide-based polymer material, a maleic anhydride-based polymer material (for example, a maleic anhydride copolymer such as methylvinylether-maleic anhydride copolymer), an acrylamide-based polymer material (for example, polyacrylamide, a block copolymer of polyglycidylmethacrylate-dimethylacrylamide (PGMA-DMAA)), water-soluble nylon, polyvinyl alcohol, polyvinylpyrrolidone, and the like.

Here, a distal portion 10 of the sheath main body 3 is formed such that the outer dimension of the sheath main body 3 becomes gradually smaller toward the distal end. That is, the outer peripheral portion or outer peripheral surface of the sheath main body 3 gradually tapers or narrows toward the distal end in order to improve penetration properties when inserting the introducer sheath 1 into a blood vessel. For this reason, there is only a small gap between the inner peripheral surface of the distal end opening portion 8 and the outer peripheral surface of the catheter K while there is a sufficient gap between the lumen 6 of the sheath main body 3 and the catheter K for blood to flow therebetween. Therefore, blood in a blood vessel cannot flow into the lumen 6 through the distal end opening portion 8. In this manner, since the inflow of blood from the distal end opening portion 8 is blocked, it is difficult to circulate blood in the lumen 6.

Therefore, in order to circulate blood in the lumen 6, a distal side slope path 11, which extends from the opening portion 4, penetrates the outer peripheral wall, and communicates with the lumen 6, is formed in the vicinity of the distal portion of the sheath main body 3, and a proximal side slope path 12, which extends from the opening portion 5, penetrates the outer peripheral wall, and communicates with the lumen 6, is formed in the vicinity of the proximal portion of the sheath main body 3. The distal side slope path 11 and the proximal side slope path 12 are inclined such that blood can flow into the lumen 6 and flow out from the lumen 6 along the blood flow in a blood vessel. That is, the distal side slope path 11 is configured so as to be inclined toward the proximal side or proximal end from the outer peripheral surface to the inner peripheral surface of the sheath main body 3, and the proximal side slope path 12 is configured so as to be inclined toward the distal side or distal end from the outer peripheral surface to the inner peripheral surface of the sheath main body 3. Stated differently, the distal side slope path 11 is a through hole that opens to the outer surface of the sheath main body 3 at a location that is distal of the location at which the through hole 11 opens to the inner surface of the sheath main body 3, and the proximal side slope path 12 is another through hole that opens to the outer surface of the sheath main body 3 at a location that is proximal of the location at which the through hole 12 opens to the inner surface of the sheath main body 3. Accordingly, in a case, for example, where blood in a blood vessel flows from the distal side to the proximal side (blood flow is in the proximal direction or to the right in FIGS. 2 and 3) of the introducer sheath 1, it is possible to make blood in the blood vessel smoothly flow into the lumen 6 through the distal side flow path 11 and to make the blood, which has been circulated in the lumen 6 from the distal side to the proximal side, smoothly flow out to the blood vessel through the proximal side slope path 12. The inclined nature of the distal side flow path 11 and the proximal side flow path 12 means that the two flow paths 11, 12 are other than (i.e., not) perpendicular to the central axis of the sheath main body 3.

The sheath main body 3 can be formed of, for example, metal such as pseudoelastic alloy, shape memory alloy, stainless steel and the like, or resin such as polyolefin, polyvinyl chloride, polyamide and the like. In addition, sheath main bodies having various lengths can be used as the sheath main body 3 in accordance with the application or use of the sheath main body, and for example, a sheath main body having a length of 10 cm 100 cm can be used.

Next, an operation of Embodiment 1 will be described.

First, the skin is punctured with a puncture needle toward a blood vessel, and a guide wire is inserted into the blood vessel through the lumen of the puncture needle. Next, the puncture needle is removed from the blood vessel while maintaining the guide wire to be indwelled in the blood vessel, and a dilator equipped with an introducer sheath is inserted into the blood vessel through the skin along the guide wire. Then, the guide wire and the dilator are removed from the inside of the blood vessel while maintaining the introducer sheath in an indwelled condition in the blood vessel, and as shown in FIG. 3, the introducer sheath 1 is indwelled in a blood vessel V. At this time, the introducer sheath 1 is inserted into or moved to a position within the blood vessel V at which the opening portion 5 formed in the vicinity of the proximal portion is positioned. Here, in a case where blood in the blood vessel V flows from the distal side to the proximal side of the introducer sheath 1, it is preferable that the opening portion 5 is positioned so as to be as far as possible on a downstream side with respect to the blood flow. That is, the opening portion 5 is positioned in the vicinity of the proximal portion in an insertion portion 13 of the introducer sheath 1 which has been inserted into the blood vessel V.

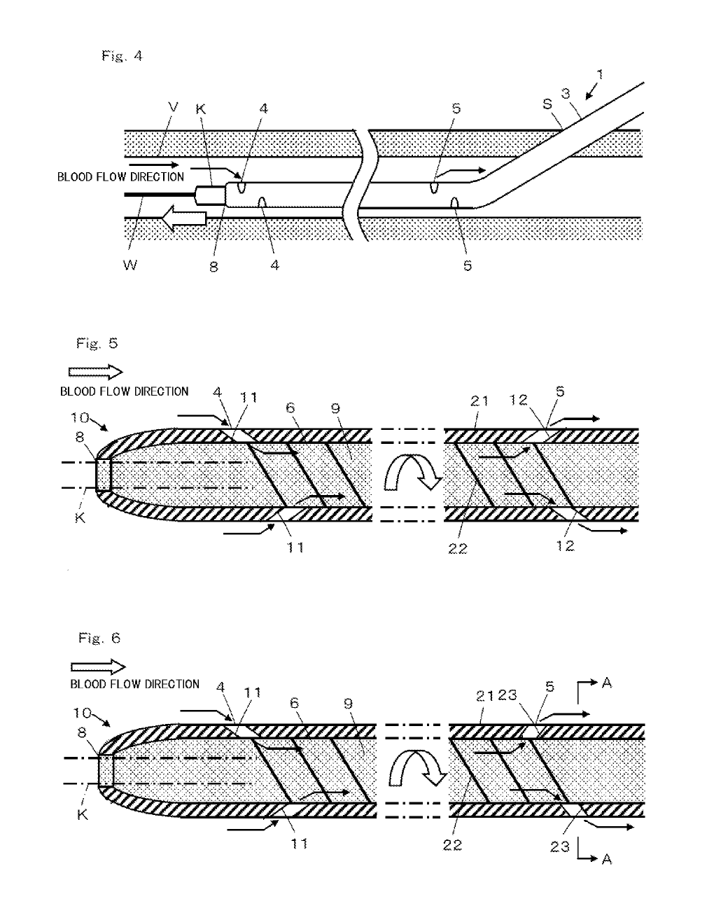

In this manner, when the introducer sheath 1 is indwelled in the blood vessel V, for example, a guide wire W and the catheter K are sequentially introduced into the port 7 which is formed in the hub 2. At this time, in a state before the catheter K is introduced into the introducer sheath 1, the distal end opening portion 8 of the sheath main body 3 is largely open and blood flows from the distal end opening portion 8 into the lumen 6. Therefore, the hydrophilic polymer 9 with which the inner peripheral surface of the sheath main body 3 is coated is wetted by blood and exhibits lubricity. For this reason, the catheter K which has been introduced from the port 7 is smoothly inserted into the distal portion of the sheath main body 3. Then, as shown in FIG. 4, the catheter K is led out to the inside of the blood vessel V from the distal end opening portion 8 of the sheath main body 3.

When the catheter K is led out of the introducer sheath and into the inside of the blood vessel from the distal end opening portion 8, as shown in FIG. 2, most of the distal end opening portion 8 is closed by the catheter K, and the sheath main body enters a state where there is only a small gap between the inner periphery of the distal end opening portion 8 and the outer periphery of the catheter K. For this reason, most of blood in the blood vessel V cannot flow into the lumen 6 through the distal end opening portion 8, and blood moving outside of the sheath main body 3 to the proximal side flows into the lumen 6 through the distal side slope path 11 from the opening portion 4 which is formed in the vicinity of the distal portion of the sheath main body 3. Because the distal side slope path 11 is inclined toward the proximal direction or toward the proximal end from the outer peripheral surface to the inner peripheral surface of the sheath main body 3, blood smoothly flows into the lumen 6 without being opposed to the blood flow in the blood vessel V. For this reason, it is possible to make blood flow into the lumen 6 from the blood vessel V without significantly reducing the force of blood.

Blood which has flowed into the lumen 6 from the distal side slope path 11 is circulated along the lumen 6 toward the proximal side or proximal end on which the proximal side slope path 12 is formed. At this time, the inner peripheral surface of the sheath main body 3 is coated with the hydrophilic polymer 9 and part of the blood circulating inside the lumen 6 is supplied to the hydrophilic polymer 9 (i.e., the blood contact the hydrophilic polymer 9). Accordingly, the hydrophilic polymer 9 is wetted by blood and lubricity is imparted to the inner peripheral surface of the sheath main body 3.

Blood which has been circulated in the lumen 6 up to the vicinity of the proximal portion of the sheath main body 3 in this manner flows out into the blood vessel V from the opening portion 5 through the proximal side slope path 12 which is located in the vicinity of the proximal portion of the sheath main body 3. The proximal side slope path 12 is inclined in the distal direction or toward the distal side (distal end) from the outer peripheral surface to the inner peripheral surface of the sheath main body 3, and therefore, it is possible to make blood smoothly flow out into the blood vessel V without going against the flow of the blood flow in the lumen 6.

In this manner, even in a state where inflow of blood from the distal end opening portion 8 is blocked due to the catheter K led out from the distal end opening portion 8 of the sheath main body 3, blood is permitted to relatively smoothly flow into the lumen 6 from the opening portion 4 in the vicinity of the distal portion of the sheath main body 3 and is able to relatively smoothly flow out into the lumen 6 from the opening portion 5 which is located in the vicinity of the proximal portion of the sheath main body 3. Therefore, it is possible to continuously circulate blood in the lumen 6. For this reason, blood is continuously supplied to the hydrophilic polymer 9 with which the inner peripheral surface of the sheath main body 3 is coated, and therefore, it is possible to maintain the inner peripheral surface of the sheath main body 3 in a lubricated state.

By virtue of the inner peripheral surface of the sheath main body 3 exhibiting a lubrication state, even after the catheter K is led out into the blood vessel from the distal end opening portion 8 of the sheath main body 3, the catheter K can promptly advance into the blood vessel V along the guide wire W without receiving large sliding resistance from the inner peripheral surface of the sheath main body 3. Then, the distal portion of the catheter K is delivered to a target diseased region and treatment of the diseased region is performed using the catheter K. Even during this treatment operation, since the catheter K does not receive large sliding resistance, it is possible to precisely move the catheter K and to perform accurate treatment on the diseased region.

According to the present embodiment, it is possible to make blood flow into the lumen 6 and to make the blood flow out from the lumen 6 along the blood flow in the blood vessel V. Therefore, it is possible to smoothly perform inflow and outflow of blood and to continuously circulate blood in the lumen 6. For this reason, it is possible to maintain the inner peripheral surface of the sheath main body 3 at a lubrication state by maintaining the hydrophilic polymer 9, with which the inner peripheral surface of the sheath main body 3 is coated, in a wetted state. Accordingly, it is possible to suppress sliding resistance and to perform accurate treatment on the diseased region in the blood vessel V, through a series of operations of the catheter K.

It is possible to continuously circulate blood in the lumen 6 by virtue of the distal side slope path 11 and the proximal side slope path 12 in the sheath main body 3. Therefore, it is possible to prevent the generation of a thrombus by suppressing stagnation of blood in the lumen 6. For this reason, it is possible to obtain a sufficient effect even in a case where the distal side slope path 11 and the proximal side slope path 12 are provided in a usual introducer sheath in which the inner peripheral surface of the sheath main body 3 has not been coated with the hydrophilic polymer 9.

In addition, the distal side slope path 11 and the proximal side slope path 12 are formed such that blood is able made to smoothly flow into the lumen 6 and to smoothly flow out from the lumen 6. Therefore, it is unnecessary to form many through-holes in the sheath main body 3, and at least one distal side slope path 11 and at least one proximal side slope path 12 may be formed in the sheath main body. In this manner, it is possible to maintain the strength of the sheath main body 3 by minimizing the number of through-holes formed in the sheath main body 3. Accordingly, it is possible to prevent kinking of the sheath main body, for example, when being introduced into the skin or a blood vessel and to prevent increase in resistance during insertion of a catheter into an introducer sheath.

### Embodiment 2

A spiral groove may be further formed on the inner peripheral surface of the sheath main body 3 of Embodiment 1 such that blood circulates while drawing a spiral orbit in the lumen 6.

For example, as shown in FIG. 5, it is possible to form a spiral groove 22 continuously extending from the distal side slope path 11 to the proximal side slope path 12 on the inner peripheral surface of a sheath main body 21. By forming the groove 22 between the distal side slope path 11 and the proximal side slope path 12 in this manner, blood, which has flowed into the lumen 6 through the distal side slope path 11 located in the vicinity of the distal portion of the sheath main body 21, circulates toward the proximal side (proximal end) while drawing a spiral orbit or flowing along a spiral path in the lumen 6. For this reason, blood circulating inside the lumen 6 also moves to the outside, that is, in a radial outward direction of the sheath main body 21 due to centrifugal force while advancing toward the proximal side or proximal end. Accordingly, blood in the lumen 6 circulates toward the proximal end while being spread on the inner peripheral surface side of the sheath main body 21 and flows out into the blood vessel V through the proximal side slope path 12 when circulating blood has reached to the proximal portion of the sheath main body 21.

According to the present embodiment, blood in the lumen 6 circulates on the proximal side while being made to move to the inner peripheral surface side by virtue of the spiral groove 22 on the inner peripheral surface of the sheath main body 21. Therefore, it is possible to further increase the outflow rate of blood from the proximal side slope path 12. Accordingly, the flow rate of blood flowing in the lumen 6 increases, and therefore, it is possible to continuously circulate blood in the lumen 6 even if the length of the introducer sheath is relatively long or the like, and to maintain lubricity by continuously wetting the hydrophilic polymer 9 with which the inner peripheral surface of the sheath main body 21 is coated.

In this Embodiment 2, it is possible to form at least either of the distal side slope path 11 and the proximal side slope path 12 to be more inclined in a circumferential direction of the sheath main body 21 along the spiral groove 22.

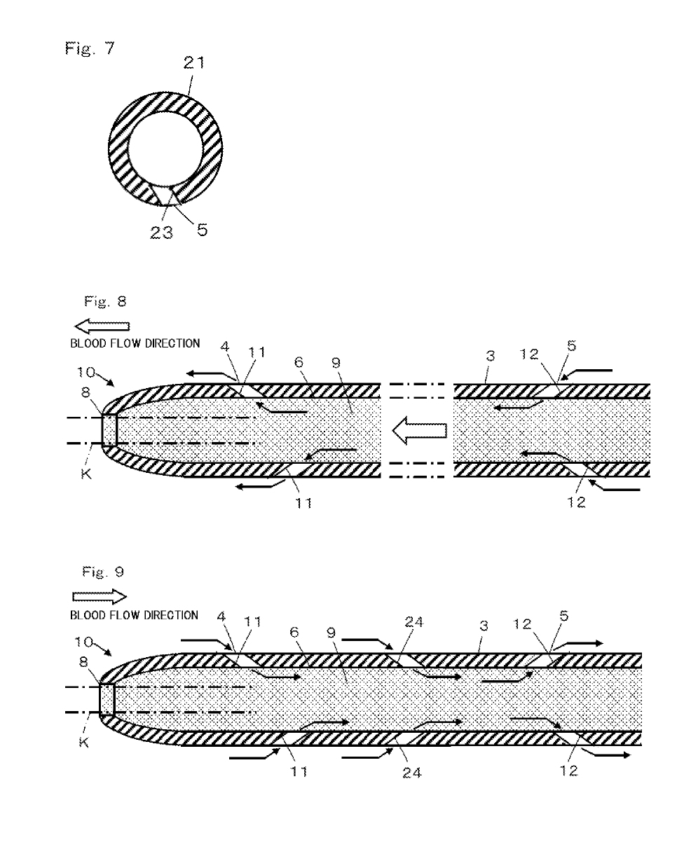

For example, as shown in FIGS. 6 and 7, it is possible to form a proximal side slope path 23 in the vicinity of the proximal portion of the sheath main body 21. The proximal side slope path 23 is inclined toward the distal side or distal end (in the distal direction) from the outer peripheral surface to the inner peripheral surface of the sheath main body 21 and is also inclined in the circumferential direction of the sheath main body 21 along the groove 22. Thus, in this embodiment, the central axis of the proximal side slope path 23 does not pass through (i.e., does not intersect) the longitudinally extending central axis of the sheath main body 21. That is, the proximal side slope path 23 is inclined in the radial direction and in the circumferential direction of the sheath main body 21, and is formed on an extension line of the groove 22. By forming the proximal side slope path 23 along the spiral groove 22 in this manner, it is possible to cause blood, which circulates while flowing along a spiral orbit from the distal side to the proximal side in the lumen 6, flow out into the blood vessel V through the proximal side slope path 23 along the orbit (as a continuation of the spiral orbit or spiral flow of the blood), and to more smoothly make the blood flow out from the lumen 6.

By forming the spiral groove on the inner peripheral surface of the sheath main body as in the above-described Embodiment 2, the flexibility of the sheath main body is also improved and the sheath main body portion of the introducer sheath is more easily bent along a blood vessel when being introduced into the blood vessel.

In the above-described Embodiments 1 and 2, the introducer sheath is inserted into the blood vessel V such that the distal side or distal end of the introducer sheath is positioned upstream with respect to the blood flow direction in the blood vessel V. However, it is also possible to obtain the same effect even if the introducer sheath is inserted into the blood vessel V such that the distal end of the introducer sheath is positioned downstream with respect to the blood flow direction in the blood vessel V.

FIG. 8 shows an example in which the introducer sheath 1 of Embodiment 1 is inserted into the blood vessel V such that the distal end of the introducer sheath is positioned downstream with respect to the blood flow direction in the blood vessel V. In this manner, blood in the blood vessel V smoothly flows into the lumen 6 by way of the opening portion 5, which is located in the vicinity of the proximal portion of the sheath main body 3, through the proximal side slope path 12, and the blood, which has circulated in the lumen 6 from the proximal end to the distal end, smoothly flows out into the blood vessel V from the opening portion 4 through the distal side slope path 11 which is located in the vicinity of the distal portion of the sheath main body 3. That is, in a case where the introducer sheath 1 is disposed such that the distal end of the introducer sheath is downstream with respect to the blood flow direction, only the circulation direction of blood in the lumen 6 is reversed, and therefore, it is unnecessary to change the structure of the introducer sheath.

In this manner, blood is made to flow into the lumen and flow out from the lumen along the blood flow in the blood vessel V, and therefore, it is possible to smoothly perform the inflow and the outflow of blood.

In addition, in the above-described Embodiments 1 and 2, the distal side slope path is located in the vicinity of the distal portion of the sheath main body. However, it is possible to form the distal side slope path over a wide range from the vicinity of the distal portion to the vicinity of the central portion of the sheath main body. In addition, in the above-described Embodiments 1 and 2, the proximal side slope path is located in the vicinity of the proximal portion of the sheath main body. However, it is possible to position the proximal side slope path over a wide range from the vicinity of the proximal portion of the sheath main body to the vicinity of the central portion of the sheath main body. For example, as shown in FIG. 9, it is possible to further form a proximal side slope path 24, which may be inclined toward the proximal end (in the proximal direction) from the outer peripheral surface to the inner peripheral surface, in the vicinity of the central portion of the sheath main body 3 in the introducer sheath 1 of Embodiment 1. Accordingly, the inflow rate of blood into the lumen 6 increases, and therefore, it is possible to maintain circulation of blood in the lumen 6 even when, for example, the length of the introducer sheath is relatively long.

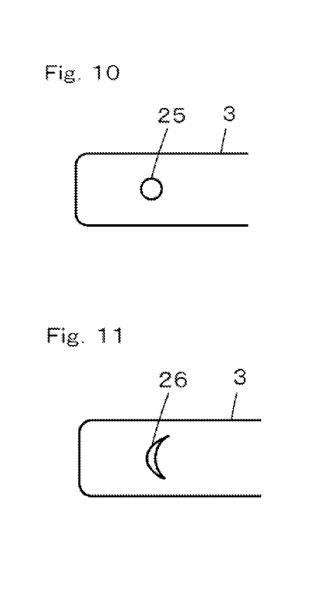

In addition, in the above-described Embodiments 1 and 2, the opening portions 4 and 5 are elliptically-shaped. However, the invention is not limited to this shape as long as blood can flow into the lumen and flow out from the lumen along the blood flow in the blood vessel V. For example, as shown in FIG. 10, it is possible to configure each of the opening portions as a circular opening portion 25 in the sheath main body 3. In addition, as shown in FIG. 11, it is possible to configure each of the opening portions as a crescent-shaped opening portion 26 in the sheath main body 3. Although not shown in the drawing, a square opening portion or a rectangular opening portion may be formed in the sheath main body.

In addition, in the above-described Embodiments 1 and 2, the introducer sheath has been used as a sheath. However, the present invention is not limited to such use as long as the sheath is inserted into the blood vessel V and blood flows into the lumen. For example, it is possible to form a distal side slope path in the vicinity of a distal portion and to form a proximal side slope path in the vicinity of a proximal portion, with respect to a guiding catheter. Accordingly, it is possible to continuously circulate blood in the guiding catheter and to improve slidability of the catheter or dilator which is inserted into the guiding catheter by, for example, coating the inner peripheral surface of the guiding catheter with a hydrophilic polymer.

The detailed description above describes embodiments of a sheath representing examples of the inventive sheath disclosed here. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

1. A sheath comprising:
an elongated sheath main body insertable into a blood vessel, the sheath main body possessing a distal portion at a distal end of the sheath main body and a proximal portion at a proximal end of the sheath main body, the sheath main body possessing an outer peripheral surface;
the sheath main body including a lumen so that the sheath main body possesses an inner peripheral surface;
at least one distal side slope path passing through an outer peripheral wall of the sheath main body in a vicinity of the distal portion of the sheath main body;
at least one proximal side slope path passing through the outer peripheral wall of the sheath main body in a vicinity of a proximal portion of the sheath main body;
in order to make blood flow into the lumen and to make blood flow out from the lumen along the blood flow in a blood vessel, the at least one distal side slope path being inclined toward the proximal end of the sheath main body from the outer peripheral surface of the sheath main body to the inner peripheral surface of the sheath main body and the at least one proximal side slope path being inclined toward the distal end of the sheath main body from the outer peripheral surface of the sheath main body to the inner peripheral surface of the sheath main body.

2. The sheath according to claim 1, wherein the inner peripheral surface of the sheath main body is coated with a hydrophilic polymer which exhibits lubricity when wetted by blood flowing into the sheath main body.

3. The sheath according to claim 1, wherein a spiral groove is formed on the inner peripheral surface of the sheath main body so that the blood which has flowed into the lumen circulates while flowing along a spiral flow path.

4. The sheath according to claim 3, wherein at least one of the distal side slope path and the proximal side slope path is further inclined in a circumferential direction of the sheath main body along the groove.

5. A sheath comprising:
an elongated sheath main body insertable into a blood vessel and possessing a distal portion and a proximal portion, the sheath main body possessing an outer peripheral surface;
the sheath main body including a lumen passing completely through the lumen, the sheath main body including an inner peripheral wall surrounding the lumen;
a first through hole passing through the outer peripheral wall of the sheath main body so that the first through hole opens to the outer peripheral surface of the sheath main body and opens to the inner peripheral surface of the sheath main body, the first through hole opening to the outer peripheral surface of the sheath main body at a location more distal of a location at which the first through hole opens to the inner peripheral surface of the sheath main body;
a second through hole passing through the outer peripheral wall of the sheath main body so that the second through hole opens to the outer peripheral surface of the sheath main body and opens to the inner peripheral surface of the sheath main body, the second through hole opening to the outer peripheral surface of the sheath main body at a location more proximal of a location at which the second through hole opens to the inner peripheral surface of the sheath main body; and
the first through hole being located distal of the second through hole.

6. The sheath according to claim 5, wherein the inner peripheral surface of the sheath main body is coated with a hydrophilic polymer which exhibits lubricity when wetted by blood flowing into the sheath main body.

7. The sheath according to claim 6, wherein the inner peripheral surface of the sheath main body includes a spiral groove that causes blood which has flowed into the lumen to flow along a spiral flow path.

8. The sheath according to claim 7, wherein the first through hole is inclined in a circumferential direction of the sheath main body.

9. The sheath according to claim 8, wherein the second through hole is inclined in a circumferential direction of the sheath main body.

10. The sheath according to claim 7, wherein the second through hole is inclined in a circumferential direction of the sheath main body.

11. The sheath according to claim 5, wherein the inner peripheral surface of the sheath main body includes a spiral groove that causes blood which has flowed into the lumen to flow along a spiral flow path.

12. The sheath according to claim 11, wherein the first through hole is inclined in a circumferential direction of the sheath main body.

13. The sheath according to claim 12, wherein the second through hole is inclined in a circumferential direction of the sheath main body.

14. The sheath according to claim 11, wherein the second through hole is inclined in a circumferential direction of the sheath main body.

15. The sheath according to claim 5, wherein there are plural first through holes passing through the outer peripheral wall of the sheath main body.

16. A method of using a sheath comprising:
inserting the sheath into a blood vessel, the sheath comprising: an elongated sheath main body possessing a distal end, a proximal end and an outer peripheral surface, the sheath main body including a lumen so that the sheath main body possesses an inner peripheral surface, a distal side slope path passing through the outer peripheral wall of the sheath main body and being inclined toward the proximal end of the sheath main body from the outer peripheral surface of the sheath main body to the inner peripheral surface of the sheath main body, and a proximal side slope path passing through the outer peripheral wall of the sheath main body and being inclined toward the proximal end of the sheath main body from the outer peripheral surface of the sheath main body to the inner peripheral surface of the sheath main body and the at least one proximal side slope path being inclined toward the distal end of the sheath main body from the outer peripheral surface of the sheath main body to the inner peripheral surface of the sheath main body; and
moving the sheath within the blood vessel so that blood in the blood vessel either enters the lumen by way of the distal side slope path and exist the lumen by way of the proximal side slope path, or enters the lumen by way of the proximal side slope path and exits the lumen by way of the distal side slope path.

17. The method according to claim 16, wherein the blood entering the lumen wets a hydrophilic polymer on the inner peripheral surface of the sheath main body so that the inner peripheral surface of the sheath main body exhibits lubricity.

18. The method according to claim 16, further comprising causing the blood entering the lumen to circulate in the lumen along a spiral flow path.

19. The method according to claim 16, wherein the moving of the sheath within the blood vessel includes moving the sheath along a guide wire previously positioned in the blood vessel, the guide wire guiding movement of the sheath in the blood vessel.
